(19) United States
(12) Patent Application Publication
(10) Pub. No.: US 2021/0369901 A1
(43) Pub. Date: Dec. 2, 2021

(54) MOBILE DEVICE HAVING AN INTEGRATED DISINFECTING DEVICE FOR AIRCRAFT CABINS

(71) Applicants: Andrew MUIN, Hamburg (DE); Stephan SONTAG, Hamburg (DE)

(72) Inventors: Andrew MUIN, Hamburg (DE); Stephan SONTAG, Hamburg (DE)

(21) Appl. No.: 17/330,864

(22) Filed: May 26, 2021

(30) Foreign Application Priority Data
May 28, 2020 (DE) 102020114395.8

(51) Int. Cl. A61L 2/22; B64D 11/00; A61B 5/01

(52) U.S. Cl. CPC A61L 2/22; B64D 11/00; A61B 2560/0431; A61L 2202/16; A61L 2202/25; A61B 5/01

(57) ABSTRACT

A mobile device having an integrated disinfecting device for aircraft cabins, having a base having an upper side and, on a lower side, rotatably mounted wheels for moving the base on a floor; a frame fastenable to an upper side of the base; a dispenser unit which is coupled to the frame and configured to discharge on demand disinfectant in a metered manner at a front side; a supply unit coupled to the frame and configured to provide at least the disinfectant; wherein the dispenser unit is configured to be movable between a stowage state and a use state; and wherein the frame and/or the disinfecting device in the stowage state have/has a size which does not substantially exceed a size of a full-size cabin trolley.

### CROSS-REFERENCES TO RELATED APPLICATIONS

This application claims the benefit of the German patent application No. 102020114395.8 filed on May 28, 2020, the entire disclosures of which are incorporated herein by way of reference.

### FIELD OF THE INVENTION

The invention relates to a mobile device having an integrated disinfecting device for aircraft cabins. The invention furthermore relates to an aircraft having a cabin with at least one storage space for receiving a device of this type.

### BACKGROUND OF THE INVENTION

Travel in commercial aircraft is usually associated with a space-optimized design of passenger cabins in which passengers sit closely next to one another in a tight space. Surfaces, for example seat faces, storage compartments and backrests, are frequently touched. In such a situation there may be the overall risk for viruses to be propagated by infection by way of smears or droplets. Since there is nevertheless a certain demand for mobility, it is desirable for travel to be made possible despite the above, whereby special hygiene measures should be considered.

To this end, it is known for dispensers for disinfectant to be provided at strategically expedient locations in public buildings, for example airports. It is moreover known for disinfectant to be carried along in personal items of baggage and to be used when required.

### SUMMARY OF THE INVENTION

It is an object of the invention to achieve a device or system which can be used, in particular in aircraft cabins, and which enhances the level of hygiene onboard independently of any personal carry-on disinfectants, and nevertheless does not require a large installation space.

Proposed is a mobile device having an integrated disinfecting device for aircraft cabins, having a base having an upper side and, on the lower side, rotatably mounted wheels for moving the base on a floor; a frame which is able to be fastened to an upper side of the base; a dispenser unit which is coupled to the frame and configured for discharging on demand a disinfectant in a metered manner at a front side; and a supply unit which is coupled to the frame and configured for providing at least the disinfectant; wherein the dispensing unit is configured for being moved to a stowage state and a use state; and wherein the frame and/or the device in the stowage state have/has a size which does not substantially exceed the size of a full-size cabin trolley. A full-size cabin trolley may preferably have the standard dimensions 80.5×30×103 cm (L×W×H). A configuration as a half-size trolley or else as a smaller transport box or a standard container box is also possible.

The device according to the invention permits a disinfectant to be used on demand directly in an aircraft cabin in that a compact, mobile unit which can be set up within the passenger cabin is provided. The device can be disposed in the trolley storage space during the flight. The device can be easily relocated to a door region and set up there before boarding the aircraft cabin.

The frame of the device is provided for pre-defining an external shape of the device and, in particular, for permitting ergonomic handling. To this end, the frame may have various elements which define the external shape of the frame. Inter alia, a frame which has a plurality of posts and is closed by cladding elements and defines a substantially cuboid space could be provided. The frame can be fastened to an upper side of the base such that the unit, composed of the base having rotatably mounted wheels and the frame having installations disposed thereon, can be easily moved in the aircraft cabin.

The supply unit could be disposed in the frame and have at least one disinfectant. The supply unit is coupled to the aforementioned dispenser unit and provides the latter with the disinfectant.

The dispenser unit is provided for discharging on demand the disinfectant in a metered manner on the front side. It is expedient for the discharging to function in a non-contacting manner so as to minimize any physical contact with the device. This means that a user can activate a sensor switch which initiates the discharge of the disinfectant. The sensor could be situated at a discharging point and, for example, respond directly to a hand moved toward the sensor. The dispenser unit is preferably able to be operated electrically such that a user does not have to do perform any manual pumping procedure. To this end, a battery or any other suitable energy accumulator could be provided in the frame and/order in the dispenser unit.

The dispenser unit is furthermore configured to be moved to a stowage state. The unit composed of the dispenser unit and the frame is particularly compact, in particular in the stowage state, and has external dimensions which are smaller than in the use state. Conversely, the use state is provided for permitting an ideally ergonomic handling of the dispenser unit. Consequently, a user does not have to bend down or look for a discharging point of the dispenser unit but should be able to initiate any disinfecting as intuitively as possible.

The stowage of the device in a trolley storage space is particularly expedient because the mobile device in this instance can be carried onboard the aircraft and used on an outbound and inbound flight on short-haul flights, for example. Cost-intensive handling of replaceable devices which have to be provided outside the aircraft can be avoided as a result.

In one advantageous embodiment, the dispenser unit is disposed so as to be vertically movable on the frame, wherein the dispenser unit in the stowage state has a short as possible spacing from the base, and in the use state is in an elevated position so as to be spaced apart from the base. To this end, the frame could have a linear guide, for example, which has one guide rail or two guide rails that are disposed so as to be mutually parallel and spaced apart from one another. The dispenser unit, in this instance, could be pushed into a lower position in which the dispenser unit is at least approximately directly adjacent to the base. The dispenser unit in an upper position is significantly spaced apart from the base and permits an ergonomic position to be assumed for the handling or the discharging of the disinfectant. It is conceivable for the dispenser unit to have a spring mechanism for simplifying the movement, the spring mechanism being configured for at least partially compensating the weight of the dispenser unit. It may be expedient for a gas spring to be used to this end.

In one advantageous embodiment, at least one of the wheels is coupled to a drive device. The handling of the device is significantly facilitated as a result, as it could be cumbersome to move the device along an aisle in the cabin, depending on the load and the scope of functions. To this end, at least one of the wheels can be driven, for example by an electric motor which is integrated in a wheel hub or coupled to the wheel by way of a shaft. There could be a control unit which for actuating the drive device has input means for activating or deactivating the drive. The base could moreover also be designed for autonomous driving.

In one further, advantageous embodiment, the dispenser unit has at least one recess for inserting a hand and a sensor unit for identifying the inserted hand, wherein a discharge device which is coupled to the sensor unit is configured for spraying the disinfectant into the respective recess. To this end, the at least one recess is disposed on the front side and could be situated somewhat above hip height. A user standing in front of the front side can intuitively insert his/her hands in to the one, preferably two, recesses, whereby the sensor unit identifies the presence of a hand and only then sprays the disinfectant.

The at least one recess is preferably closable. A cover which can be moved so as to sit flush in an opening contour of the recess could be provided for closing. The cover could be provided so as to be displaceable or pivotable. In particular in a stowage position, this may prevent foreign matter making its way into the respective recess and leading to any malfunction in the latter.

It is advantageous for the device to furthermore be provided with a protective screen which is disposed so as to be vertically movable on the device and which is movable to a retracted and a deployed position, wherein the protective screen in the deployed position protrudes beyond the dispenser unit. The protective screen could, in particular, be disposed behind the dispenser unit whereby a disposal in front of the dispenser unit or above the dispenser unit is however also conceivable. The protective screen could, in particular, be embodied so as to be transparent, for example in the form of a Plexiglas pane. This is however not mandatory. The protective screen serves for achieving protection for people directly behind the mobile device in relation to an infection by droplets. For example, crew members could be present behind the mobile device and monitor the use of the mobile device. If the protective screen is in a deployed position, the risk of an infection by droplets can be significantly minimized even at a comparatively minor spacing from the device.

The device may furthermore have an infrared temperature measurement unit which is configured for detecting an elevated body temperature of a passenger at the dispenser unit. The mobile device could be disposed, for instance, in a door region of an aircraft cabin such that each passenger boarding the aircraft cabin is able to disinfect his/her hands. The body temperature of each user could simultaneously be checked by an infrared measuring apparatus during this procedure so that people with an elevated body temperature can be identified. In the case of infections by viruses which lead to an elevated body temperature, a corresponding indication could be provided to the crew. The respective passenger may optionally be denied access.

In one advantageous embodiment, the device furthermore has a temperature display unit which is coupled to the infrared temperature measurement unit and which is disposed on a side of the frame that lies opposite the front side. When monitoring the use of the mobile device, a person standing behind the dispenser unit could directly receive an indication pertaining to a potentially elevated body temperature of a passenger. It is particularly expedient for this embodiment to be combined with the aforementioned protective screen so as to enable a minimized spacing which provides sufficient privacy.

In one advantageous embodiment the frame, for separately stowing the frame, is able to be releasably coupled to the base. The frame may be embodied so compact that the frame is also able to be accommodated in a stowage compartment in the aircraft cabin without the base. It could also be made possible for a replacement of a frame, or the unit composed of the frame and the dispenser unit, to be carried out should a defect or a malfunction arise. Independently thereof, it is nevertheless expedient for the dimensions of the entire device in a stowage state to be limited to the aforementioned size.

It is particularly preferable for the supply unit to be configured for receiving a replaceable container with disinfectant. A separate container can be refilled outside the aircraft cabin and rapidly replaced.

It may be possible for standard containers of manufacturers of disinfectants to be used, which can be incorporated in the respective receptacle of the dispenser unit or of the frame. Specific screw connectors, needle valves, hose connectors, or other flanges can be used to this end.

The device can furthermore have a drain on the lower side of the recess, wherein the drain is able to be coupled to a container for receiving liquid waste. Since the disinfectant when discharged is often not completely taken up by the hands, small amounts can remain in the dispenser unit and, caused by gravity, accumulate at the lower end of the recess. A drain which then supplies the liquid waste to a further container is expedient in order to prevent excessive amounts of disinfectant from accumulating there.

The supply unit may have at least one closable compartment for providing gloves, masks, absorbent cloths, and other items. The mobile device accordingly forms a central point for adhering to hygiene regulations and can accommodate all items required to this end. For example, if passengers are travelling with an unsuitable mask or entirely without a mask, such a mask can be retrieved from the supply unit and handed out. Absorbent cloths can moreover be provided for disinfecting surfaces within the passenger cabin, the absorbent cloth being soaked with the disinfectant so as to then disinfect the respective surfaces.

In one particularly advantageous embodiment, the frame and the base are mutually rotatable by at least 90° about an axis which is perpendicular to the upper side of the base. The base and the frame may thus have an advantageous mutual arrangement which utilizes the available space in an optimal manner as well as permits the dispenser unit to be aligned in an expedient manner without maneuvering the base.

The base may have a pivotably mounted handlebar for guiding the base. The handlebar may be moved to a substantially vertical parking position and to a use position which runs so as to be oblique in relation to the base. A hand grip, by way of which the base can be pulled or pushed, may be disposed at one end of the handlebar. If the base is equipped with a drive device, an input unit could be disposed on the handlebar so as to initiate or suppress a driving action. This can be implemented in the form of buttons or sliding switches, for instance.

The invention finally also relates to an aircraft having a cabin with at least one storage space for receiving a mobile device as outlined above. The storage space may be situated, for example, in a galley, in particular a galley which is situated at a door region or at the aft of the aircraft.

### BRIEF DESCRIPTION OF THE DRAWINGS

Further features, advantages and potential applications of the present invention are derived from the description hereunder of the exemplary embodiments and from the figures. Here, all of the features described and/or depicted in the figures, alone or in any desired combination, form the subject matter of the present invention, independently of the combination of the features in the claims or the back-references thereof. Furthermore, identical reference signs in the figures represent identical or similar objects.

FIGS. 1 to 7 show the mobile device in various views;

FIG. 8 shows the embodiment of the frame and of the dispenser unit as a module which can be separated from the base;

FIG. 9 shows the measuring of the body temperature by means of an infrared measurement unit;

FIG. 10 shows replaceable containers for disinfectant;

FIGS. 11 and 12 show the discharging of disinfectant and the receiving of liquid waste (only FIG. 11);

FIG. 13 shows the storing of masks and the like;

FIG. 14 shows an aircraft having a cabin which is disposed therein and in which the mobile device can be used.

### DETAILED DESCRIPTION OF THE PREFERRED EMBODIMENTS

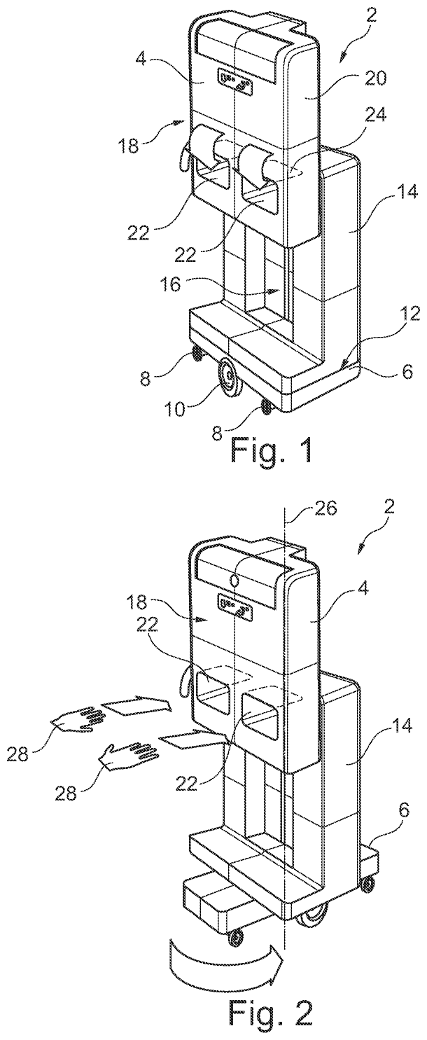

FIG. 1 shows a mobile device 2 having an integrated disinfecting device 4 for aircraft cabins. The device 2 has a base 6 which has rotatably mounted wheels 8 and 10 which stand on a floor and permit the base 6 to be moved. The base 6 has an upper side 12 to which a frame 14 is able to be fastened.

The dispenser unit 4 is disposed on the frame 14 and can be vertically moved. In the illustration in FIG. 1, the dispenser unit 4 is in a use state and has a largest possible spacing from the base 6. To this end, the frame 14 possesses a linear guide 16 on which the dispenser unit 4 is vertically guided.

The dispenser unit 4 is configured for discharging disinfectant in a metered manner at the front side 18. To this end, the dispenser unit 4 has a housing 20 which possesses recesses 22 which lie next to one another on the front side 18. One sensor unit (not shown here) can be seen in each of these recesses 22, the sensor units detecting a hand of a user being inserted. Moreover, a discharge device, for instance a spray device (not shown) which discharges the disinfectant in a metered manner on demand is disposed in each recess. In order to avoid contamination, the two recesses 22 each are able to be closed by a flap 24. The flaps 24 should be closed, in particular, when the dispenser unit 4 is situated in a lower position and the mobile device 2 consequently assumes a stowage state.

It can be seen in FIG. 2 that for aligning the frame 14, and thus the dispenser unit 4, in an optimal manner, the frame 14 may be mounted on the base 6 so as to be rotatable about a vertical rotation axis 26. For example, the mobile device 2 in the stowage state may be situated in a trolley storage space which is disposed in an aft region of the aircraft cabin. The device 2 for use is then moved to the front, along a central aisle along the longitudinal axis of the aircraft, and parked in a door region. Since the doors are disposed laterally next to the aisle, the passengers board the aircraft cabin transversely to the longitudinal axis. Once the device 2 has been parked, the frame 14 may consequently be rotated by 90° in relation to the base 6 such that each passenger directly approaches the front side 18. The passenger then inserts his/her hands 28 into the at least one recess 22 and receives a metered discharge of the disinfectant.

FIG. 3 shows the mobile device 2 in the use state thereof. The recesses 22 here are still closed by the flaps 24. A vertically displaceable protective screen 32 is disposed on the rear side 30 of the dispenser unit 4. The protective screen 32 can likewise be moved to a use position which can be seen in FIG. 3. As a result, a person standing behind the device 2 can be protected in relation to an infection by droplets from a person standing in front of the front side 18.

FIG. 4, in contrast, shows the mobile device 2 in a stowage state. The dispenser unit 4 here is moved as far down as possible such that the mobile device has dimensions which correspond at most to those of a full-size trolley. The device 2 can consequently be introduced into a storage space which is provided with cabin trolleys configured so as to be of standard size.

The mobile device 2 is shown in another view in FIG. 5, seen in the direction toward the front side 18. The device 2 is situated in the stowage state. A handlebar 34, which here is disposed in the vertical parking position, is disposed on the right side on the end side of the base 6. The handlebar 34, on the upper side thereof that faces away from the base 6, has a hand grip 36 which can be gripped by a user in order for the device 2 to be relocated.

The handlebar 34 is shown in a use position in FIG. 6, whereby the frame 14 here is rotated relative to the base 6 about the rotation axis 26 and is situated in a use state.

FIG. 7 shows the device 2 in the use state as shown in FIG. 1, seen in the direction toward the front side 18. The linear guide 16 can be better seen here. Moreover to be seen is a pivot bearing 38 for rotating the frame 14 on the base 6. The linear guide 16 may have a rail 40 which is equipped with a row of engagement elements 42. The latter are implemented as recesses or depressions, a correspondingly shaped gear wheel which is situated in the dispenser unit being able, for example, to engage in the recesses or depressions. As a result, the disinfecting unit 4 can be relocated in a motorized manner along the guide rail 40. The gear wheel then engages in the engagement elements 42 and can be rotated by a drive. It is expedient here for this drive to be implemented in a self-locking manner, for example by a spindle drive or similar, such that the assumed position of the disinfecting unit 4 is automatically maintained. However, locking devices which can be used for retaining and releasing the respective vertical position of the disinfecting unit 4 are also conceivable.

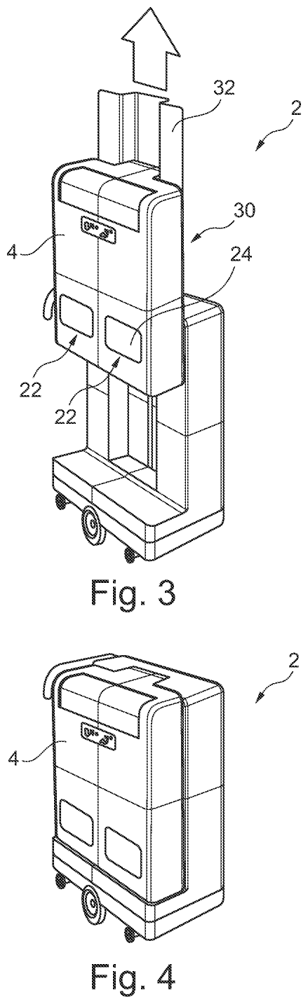

It is shown in FIG. 8 that the frame 14, having the disinfecting unit 4 disposed thereon, can be released from the base 2 and disposed as a separate module 44 in a storage space 46. A user 48 could consequently move the base 2 to the storage space 46 and then accordingly remove the module 44. The base 6 may be used for other purposes in the aircraft cabin that envisages a similar module with other functions to be received.

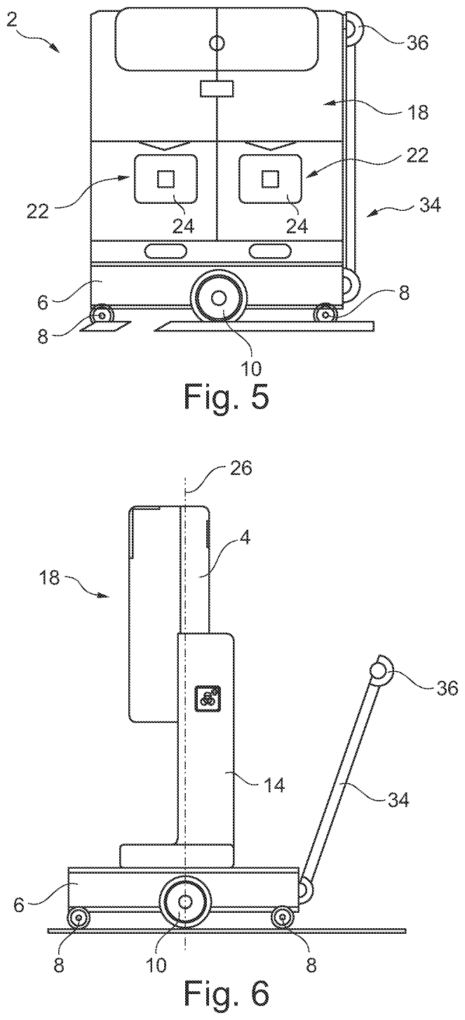

FIG. 9 shows the device 2 having an infrared temperature measuring unit 50 on the front side 18. The body temperature of a passenger 52 can be measured in a non-contacting manner by the infrared temperature measuring unit 50. A display unit 54 which is coupled to the temperature measurement unit 50 and where a user 48 can establish whether the respective passenger 52 has an elevated body temperature is disposed on the rear side 30. The display unit 54 may output, for instance, the body temperature and an all clear or warning signal. The user 48 who reads the display unit 54 is situated behind the protective screen 32 and is protected in relation to any infection by droplets.

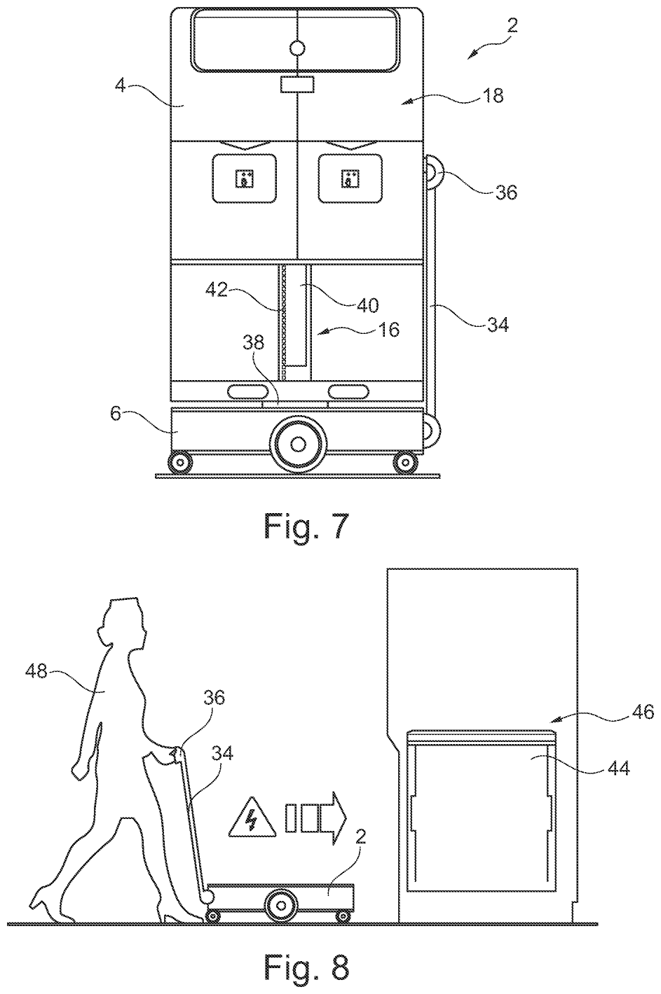

FIG. 10 shows replaceable containers 56 for disinfectant. The containers 56 can be inserted into the frame 14, more specifically into a supply unit 58. The containers 56 can be rapidly replaced when empty.

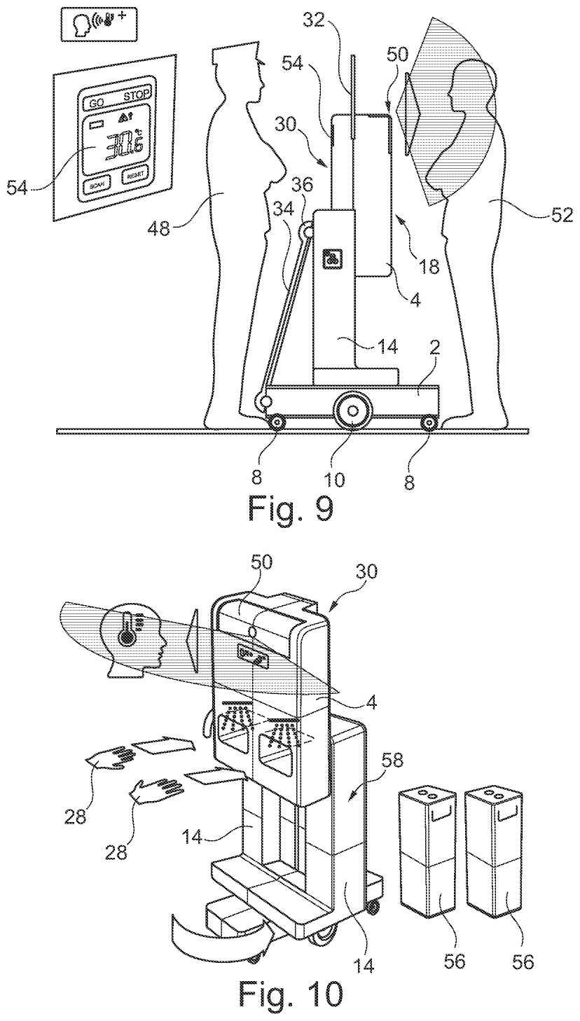

FIG. 11, in a highly schematic illustration, shows a container 56 in a supply unit 58 by means of a pump 62 being coupled to individual discharge devices in the form of spray devices 60 in the recesses 22. Excess disinfectant which accumulates in the recesses 22 can be guided through a drain 65 into a separate tank 64 for liquid waste. A pump 66 which conveys the liquid waste into the tank 64 may also be provided to this end.

FIG. 12 in a lateral view schematically shows the construction from FIG. 11, and, in particular, a disinfectant tank 56 and the pump 62. A sensor unit 61 which is provided for identifying an approaching hand so as to actuate the discharge device 60 is provided in the region of the discharge device 60.

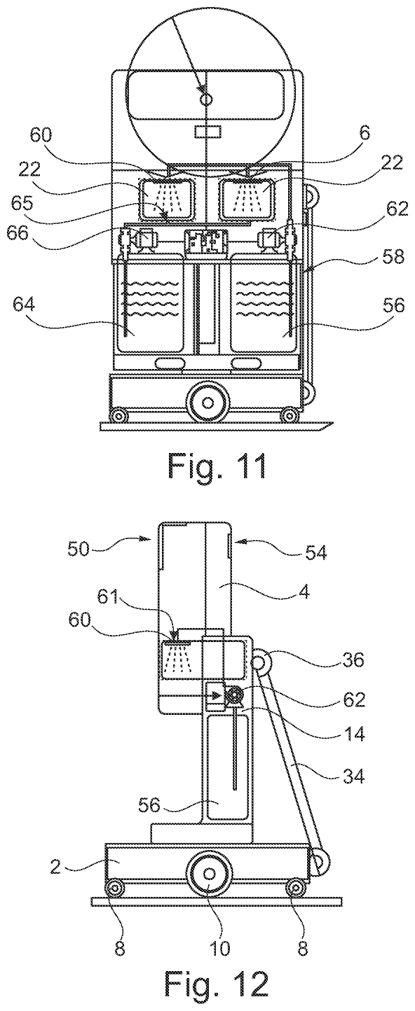

FIG. 13 indicates that masks 68 can also be accommodated in a compartment 67 in the supply unit 58. In addition to these, gloves, absorbent cloths and similar items can also be included here.

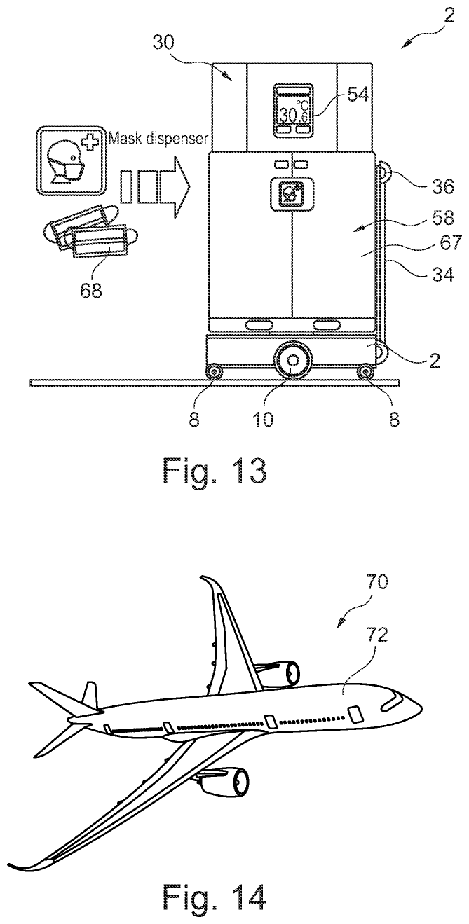

FIG. 14 finally shows an aircraft 70 which has a passenger cabin 72 into which at least one mobile device 2 of this type can be introduced.

It is additionally to be pointed out that “having” does not exclude any other elements or steps, and “a” or “one” does not exclude any multiplicity. It is furthermore to be pointed out that features which have been described with reference to one of the above exemplary embodiments can also be used in combination with other features of other exemplary embodiments described above. Reference signs in the claims are not to be considered to be limitations.

While at least one exemplary embodiment of the present invention(s) is disclosed herein, it should be understood that modifications, substitutions and alternatives may be apparent to one of ordinary skill in the art and can be made without departing from the scope of this disclosure. This disclosure is intended to cover any adaptations or variations of the exemplary embodiment(s). In addition, in this disclosure, the terms “comprise” or “comprising” do not exclude other elements or steps, the terms “a” or “one” do not exclude a plural number, and the term “or” means either or both. Furthermore, characteristics or steps which have been described may also be used in combination with other characteristics or steps and in any order unless the disclosure or context suggests otherwise. This disclosure hereby incorporates by reference the complete disclosure of any patent or application from which it claims benefit or priority.

### LIST OF REFERENCE SIGNS

2 Mobile device
4 Dispenser unit
6 Base
8 Wheel
10 Wheel
12 Upper side
14 Frame
16 Linear guide
18 Front side
20 Housing
22 Recess
24 Flap
26 Rotation axis
28 Hand
30 Rear side
32 Protective screen
34 Handlebar
36 Hand grip
38 Pivot bearing
40 Rail
42 Engagement element
44 Module
46 Storage space
48 User
50 Temperature measurement unit
52 Passenger
54 Temperature display unit
56 Container
58 Supply unit
60 Spray device/discharge device
61 Sensor unit
62 Pump
64 Tank for liquid waste
65 Drain
66 Pump
67 Compartment
68 Mask
70 Aircraft
72 Passenger cabin

1. A mobile device having an integrated disinfecting device for aircraft cabins, comprising:
a base having an upper side and, on a lower side, rotatably mounted wheels for moving the base on a floor;
a frame which is able to be fastened to an upper side of the base;
a dispenser unit which is coupled to the frame and configured to discharge on demand disinfectant in a metered manner at a front side; and
a supply unit which is coupled to the frame and configured to provide at least the disinfectant;
wherein the dispenser unit is configured to be moved between a stowage state and a use state;
wherein at least one of the frame or the overall disinfecting device in the stowage state has a size which does not substantially exceed a size of a full-size cabin trolley.

2. The mobile device according to claim 1,
wherein the dispenser unit is disposed to be vertically movable on the frame;
wherein the dispenser unit, in the stowage state, has a shortest possible spacing from the base, and in the use state lies in an elevated position to be spaced apart from the base.

3. The mobile device according to claim 1, wherein at least one of the wheels is coupled to a drive device.

4. The mobile device according to claim 1,
wherein the dispenser unit has at least one recess for inserting a hand and a sensor unit for identifying the inserted hand; and
wherein a discharge device which is coupled to the sensor unit is configured to spray the disinfectant into the recess.

5. The mobile device according to claim 4, wherein the at least one recess is closable.

6. The mobile device according to claim 1,
furthermore comprising a protective screen which is disposed to be vertically movable on the device and which is movable to a retracted and a deployed position;
wherein the protective screen in the deployed position protrudes beyond the dispenser unit.

7. The mobile device according to claim 1, furthermore having an infrared temperature measurement unit which is configured to detect an elevated body temperature of a passenger at the dispenser unit.

8. The mobile device according to claim 7, furthermore having a temperature display unit which is coupled to the infrared temperature measurement unit and is disposed on a side of the frame that lies opposite the front side.

9. The mobile device according to claim 1, wherein the frame, to accommodate separately stowing the frame, is releasably coupled to the base.

10. The mobile device according to claim 1, wherein the supply unit is configured to receive a replaceable container with the disinfectant.

11. The mobile device according to claim 4,
furthermore comprising a drain on a lower side of the recess,
wherein the drain is couplable to a container to receive liquid waste materials.

12. The mobile device according to claim 1, wherein the supply unit has at least one closable compartment to provide a storage space for gloves, masks, and absorbent cloths.

13. The mobile device according to claim 1, wherein the frame and the base are mutually rotatable by at least 90° about an axis which is perpendicular to an upper side of the base.

14. The mobile device according to claim 1, wherein the base has a pivotably mounted handlebar to guide the base.

15. An aircraft comprising:
a cabin comprising:
at least one storage space configured to receive a mobile device according to claim 1.
